(19) United States
(12) Patent Application Publication
(10) Pub. No.: US 2003/0235878 A1
(43) Pub. Date: Dec. 25, 2003

(54) Compositions and methods relating to ABCA1-mediated cholesterol efflux

(76) Inventors: Ira Tabas, New City, NY (US); Bo Feng, Teaneck, NJ (US)

Correspondence Address: John P. White, Cooper & Dunham LLP, 1185 Avenue of the Americas, New York, NY, 10036, US

(21) Appl. No.: 10/426,415

(22) Filed: Apr. 30, 2003

Related U.S. Application Data
(60) Provisional application No. 60/376,984, filed on Apr. 30, 2002.

(51) Int. Cl. C12Q 1/60

(52) U.S. Cl. 435/11

(57) ABSTRACT

This invention provides a method for determining whether an agent increases ABCA1-dependent cholesterol efflux from a cell. This invention also provides methods for increasing cholesterol efflux from a cell and for decreasing the amount of cholesterol in a cell. This invention further provides methods for increasing the likelihood that a cholesterol-loaded macrophage will survive and for decreasing the likelihood that a cholesterol-loaded macrophage will contribute to the progression of atherosclerosis. Finally, this invention provides a method for treating a subject afflicted with atherosclerosis, and a related article of manufacture.

This application claims priority of U.S. provisional application Serial No. 60/376,984, filed Apr. 30, 2002, the content of which is hereby incorporated into this application by reference.

The invention disclosed herein was made with United States government support under grant number HL-54591 and HL-56984 from the National Institutes of Health, Heart Lung and Blood Institute. Accordingly, the United States government has certain rights in this invention.

Throughout this application, various publications are referenced by author and publication date. Full citations for these publications may be found at the end of the specification immediately preceding the claims. The disclosures of these publications are hereby incorporated by reference into this application to describe more fully the art to which this invention pertains.

### BACKGROUND OF THE INVENTION

Cholesteryl ester-loaded macrophages, or foam cells, are prominent features of atherosclerotic lesions and play important roles in lesion progression (Ross et al, 1995; Libby et al, 1993). During atherogenesis, intimal macrophages internalize atherogenic lipoproteins, including modified forms of LDL, that have been retained in the arterial subendothelium (Ross et al, 1995; Tabas, 2000; Williams, 1995). This event leads directly to esterification of cellular cholesterol by acyl-coA-cholesterol acyltransferase (ACAT), resulting in “foam cell” formation (Tabas, 2000; Brown et al, 1983).

Foam cell formation can be prevented or reversed by a process known as cellular cholesterol efflux (Tall, 1998). Cholesterol efflux is the initial step of reverse cholesterol transport, a process whereby excess cholesterol in peripheral cells is delivered to the liver for excretion.

Enhancing cholesterol efflux from macrophages represents a promising strategy to promote reverse cholesterol transport and regression of atherosclerotic vascular disease.

Recently, the ATP-binding cassette transporter A1 (ABCA1) protein was shown to be an important mediator of cholesterol efflux from macrophages. Humans with full or even partial deficiency of ABCA1 have low HDL levels and increased risk for cardiovascular disease. Moreover, three reports of ABCA1 transgenic mice have shown that increased activity of ABCA1 leads to an increase in macrophage cholesterol efflux and increased reverse cholesterol transport in vivo. Thus, a potentially promising therapeutic strategy directed at atherosclerotic vascular disease is to enhance ABCA1 activity in lesional macrophages. Current strategies aimed at enhancing ABCA1 activity are directed toward increasing the cellular expression of this protein.

Macrophage death is also a prominent feature of atherosclerotic lesions (Mitchinson et al, 1996; Ball et al, 1995; Berbrerian et al, 1990; Bauriedel et al, 1997) and may affect lesion progression and/or complications. For example, death of macrophages may contribute to the release of plaque-destabilizing and thrombogenic molecules in more advanced lesions. In support of this model, “necrotic” cores of advanced atheromata, which contain the debris of dead macrophages, are located in areas predisposed to plaque rupture and acute thrombosis (Fuster et al, 1992). Moreover, fragments of plasma membrane shed by apoptotic lesional cells are rich in thrombogenic tissue factor activity (Mallat et al, 1999). More directly, apoptotic macrophages, but not apoptotic smooth muscle cells or T cells, are greatly increased in ruptured plaques versus stable plaques (Kolodgie et al, 2000), and atherectomy specimens from patients with unstable angina have approximately twice the number of dead intimal macrophage cells compared with specimens from patients with stable angina (Bauriedel et al, 1997).

Among the likely causes of lesional macrophage death is intracellular accumulation of excess free cholesterol, which is known to occur in vivo. While cholesteryl ester accumulation in lesional macrophages is often emphasized, the accumulation of free cholesterol also occurs, particularly in advanced atherosclerosis (Lundberg, 1985; Rapp et al, 1983). Presumably, this occurs because progressive lipid loading of macrophages overwhelms the cell's capacity either to esterify or efflux the free cholesterol.

### SUMMARY OF THE INVENTION

This invention provides a method for determining whether an agent increases ABCA1-dependent cholesterol efflux from a cell comprising contacting a free cholesterol-loaded cell with the agent in the presence of a cholesterol acceptor and quantitatively determining the efflux of cholesterol from the cell.

This invention also provides a method for increasing cholesterol efflux from a cell comprising contacting the cell with an agent which increases ABCA1-dependent cholesterol efflux from a cell.

This invention further provides a method for decreasing the amount of cholesterol in a cell comprising contacting the cell with an agent which increases ABCA1-dependent cholesterol efflux from the cell.

This invention further provides a method for increasing the likelihood that a cholesterol-loaded macrophage will survive comprising contacting the macrophage with an agent which increases ABCA1-dependent cholesterol efflux from a macrophage, thereby increasing the likelihood that the macrophage will survive.

This invention also provides a method for decreasing the likelihood that a cholesterol-loaded macrophage will contribute to the progression of atherosclerosis in a subject comprising contacting the macrophage with an agent which increases ABCA1-dependent cholesterol efflux from a macrophage, thereby decreasing the likelihood that the macrophage will contribute to the progression of atherosclerosis in the subject.

This invention further provides a method for treating a subject afflicted with atherosclerosis comprising administering to the subject a therapeutically effective amount of an agent which increases ABCA1-dependent cholesterol efflux from a cell, thereby treating the subject.

Finally, this invention provides an article of manufacture comprising packaging material and a pharmaceutical agent, wherein the pharmaceutical agent increases ABCA1-dependent cholesterol efflux from a cell and wherein the packaging material comprises a label indicating that the pharmaceutical agent is intended for use in treating a subject afflicted with atherosclerosis.

### BRIEF DESCRIPTION OF THE FIGURES

FIG. 1A: Cholesterol efflux to Apo-A1 is defective in free cholesterol-loaded macrophages. Mouse peritoneal macrophages were incubated for 5 h with 100 μg/ml ³H-cholesterol-labeled acetyl-LDL alone (cholesteryl ester loading) or plus 10 μg/ml of the ACAT inhibitor 58035 (free cholesterol loading). The cells were then incubated with 15 μg/ml apo-A1 for 2.5 h, and efflux of ³H-cholesterol was measured. The data are expressed as the percentage of total cellular ³H-cholesterol.

FIG. 1B: Cholesterol efflux to HDL₂ is modestly impaired in free cholesterol-loaded macrophages. Cells were treated as in FIG. 1A except following cholesterol loading the cells were incubated with 20 μg/ml HDL₂ for 2.5 h. Efflux was measured and data are presented as in FIG. 1A.

FIG. 1C: Phospholipid efflux to Apo-A1 is defective in free cholesterol-loaded macrophages. Cells were labeled for 24 h with ³H-choline chloride and then treated as in FIG. 1A, except that phospholipid efflux was measured and the data are expressed as percentage of total cellular ³H-phospholipids.

FIG. 1D: Cells were treated and cholesterol efflux was measured as in FIG. 1A, except that the time of apoA-1 incubation was varied as indicated on the x-axis.

FIG. 1E: Cells were labeled and treated as in FIG. 1C. Aliquots of free cholesterol-loaded cells were incubated for 15 min at 37° C. in the absence or presence of 0.5% or 0.2% methyl-β-cyclodextrin (CD). This treatment removes about 30% of total cellular cholesterol. All cells were then chased with media containing 15 μg/ml apoA-I for 3.33 h and phospholipid efflux was measured as in FIG. 1C.

FIG. 2A: ABCA1 protein is decreased in free cholesterolloaded macrophages. Mouse peritoneal macrophages were incubated for 5 or 7 h with 100 μg/ml acetyl-LDL in DMEM, 0.2% BSA, in the absence (CE) or presence (FC) of 58035. Aliquots of total cell protein were then subjected to immunoblot analysis for ABCA1 and the standards β-actin or β1-integrin.

FIG. 2B: Membrane-associated ABCA1 protein is decreased in free cholesterol-loaded macrophages. Cells were treated as in FIG. 2A except that aliquots of cell-surface protein instead of total protein were used for immunoblot analysis of ABCA1 expression.

FIG. 3A: ABCA1 mRNA levels are not substantially altered in free cholesterol-loaded macrophages. Mouse peritoneal macrophages were incubated for 5 h with 100 μg/ml acetyl-LDL in DMEM, 0.2% BSA, in the absence (CE) or presence (FC) of 58035. Total RNA was extracted from the cells, and the ratio of ABCA1:β-actin mRNA was determined by quantitative PCR.

FIG. 3B: Free cholesterol-loaded macrophages demonstrate increased degradation of ABCA1 protein. Macrophages were pre-incubated for 14 h with 50 μg/ml acetyl-LDL in DMEM, 0.2% BSA, in the absence (CE) or presence (FC) of 58035. The cells were then incubated for 5 h with 100 μg/ml acetyl-LDL in DMEM, 0.2% BSA, in the absence or presence of 58035, respectively, with no further additions (Control) or in the presence of 10 μg/ml cycloheximide, 50 μM ALLN, or 50 μM lactacystin as indicated. Aliquots of cell lysates were then assayed for ABCA1 and β-actin protein by immunoblot analysis.

FIG. 4A: Partial NPCl deficiency restores ABCA1-mediated cholesterol efflux in FC-loaded macrophages. Macrophages from wild-type (NPC^{+/+}) and heterozygous (NPC^{+/-}) NPC mice, all on the apoE knockout/C57 background, were incubated with medium containing 100 μg/ml ¹²⁵I-acetyl-LDL for 1, 2, 4, or 6 h, after which cholesterol esterification was assayed. In this experiment, the uptake and degradation of 125I-acetyl-LDL and in-vitro ACAT activity in the presence of excess cholesterol were similar between the two cell genotypes.

FIG. 4B: Macrophages from wild-type and heterozygous NPC mice, all on the apoE knockout/C57 background, were incubated for 5 h with medium containing 100 μg/ml ³H-cholesterol-labeled acetyl-LDL in DMEM, 0.2% BSA, in the presence of 10 μg/ml 58035. The macrophages were then incubated for 18 h in the same medium containing 15 μg/ml of apoA-I and efflux of ³H-cholesterol was measured as described in FIG. 1.

FIG. 4C: Assay was performed as in FIG. 4B, except following cholesterol loading, cells were incubated in medium containing 20 μg/ml HDL₂.

FIG. 4D: Assay was performed as in FIG. 4B, except the 18 h apoA-I incubation was done in the presence of 200 μM glyburide (GLYB) or 200 μM ortho-vanadate as indicated.

FIG. 5: Partial NPC1 deficiency restores ABCA1 protein expression in free cholesterol-loaded macrophages. Macrophages from wild-type and heterozygous NPC mice, all on the apoE knockout/C57 background, were incubated for 5 h with medium containing 100 μg/ml acetyl-LDL in DMEM, 0.2% BSA, in the absence (CE) or presence (FC) of 10 μg/ml 58035. Aliquots of total cell protein (top) or cell-surface protein (bottom) were then subjected to immunoblot analysis for ABCA1 and the standards β-actin or β1-integrin.

FIG. 6A: Low dose amphipathic amines restore ABCA1-mediated cholesterol efflux in free cholesterol-loaded macrophages. Peritoneal macrophages from C57 mice were incubated for 5 h with 100 μg/ml ³H-cholesterol-labeled acetyl-LDL in DMEM, 0.2% BSA, in the presence of 10 μg/ml 58035. The macrophages were then incubated for 6 h in the same medium containing 15 μg/ml of apoA-I in the absence or presence of the indicated concentrations of U18666A, and efflux of ³H-cholesterol was measured. The dotted line in each graph indicates the percentage of ³H-cholesterol efflux in the absence of U18666A.

FIG. 6B: Assay was conducted as in FIG. 6A, except the indicated concentrations of imipramine were used in place of U18666A.

FIG. 7A: 70 nM U18666A restores ABCA1-mediated cholesterol efflux in FC-loaded macrophages and enhances efflux in macrophages incubated long-term with acetyl-LDL. Efflux assay was conducted as described in FIG. 6A except 70 nM U18666A was used, and the apoA-I incubation time was varied as indicated.

FIG. 7B: Efflux assay was conducted as in FIG. 7A, except that 20 μg/ml HDL₂ was the cholesterol acceptor.

FIG. 7C: Macrophage cells were incubated with 100 μg/ml acetyl-LDL, without 58035, for 5 h and then incubated for a further 18 h with acetyl-LDL in the absence or presence of 70 nM U18666A.

FIG. 8: 70 nM U18666A restores the level of ABCA1 protein in free cholesterol-loaded macrophages. Macrophages were pre-incubated for 14 h with 50 μg/ml acetyl-LDL in DMEM, 0.2% BSA, in the absence (CE) or presence (FC) of 58035. The cells were then incubated for 5 h with 100 μg/ml acetyl-LDL in DMEM, 0.2% BSA, in the absence or presence of 58035, respectively, with no further additions (Control) or in the presence of 70 nM U18666A. Aliquots of total cell protein (top panel) or cell-surface protein (bottom panel) were then subjected to immunoblot analysis for ABCA1 and the standards β-actin or β1-integrin.

FIG. 9A: LDL receptor knockout mice were fed a diet containing cholesterol and saturated fat for 12 weeks in the absence or presence of 0.75 mg/kg/d U18666A (10 mice per group). Plasma was assayed for total cholesterol. Asteriks denote statistically significant differences between drug and control groups (p<0.05 by the Student's t test).

FIG. 9B: Mice were treated as in FIG. 9A and plasma was assayed for total HDL.

FIG. 9C: Mice were treated as in FIG. 9A and the proximal aorta was assayed for total atherosclerotic lesion cross-sectional area.

FIG. 9D: Mice were treated as in FIG. 9A and the proximal aorta was assayed for the area of acellular regions.

FIG. 9E: Mice were treated as in FIG. 9A and the proximal aorta was assayed for lipid core regions.

### DETAILED DESCRIPTION OF THE INVENTION

Definitions

“ABCA1” is used herein to mean “ATP-binding cassette transporter A1”, and is also referred to in the art as “ABC1”.

As used herein, “ACAT” shall mean “acyl-CoA-cholesterol acyltransferase,” which is the enzyme that catalyzes the first committed step in cholesterol ester biosynthesis. Inhibitors of this enzyme are known in the art, and are exemplified by Matsuda (1994).

“ApoA-I” shall mean “apolipoprotein A-I”, which is the major protein of high density lipoprotein (HDL).

As used herein, “cholesterol” includes, without limitation, esterified cholesterol, i.e., cholesteryl esters, and non-esterified cholesterol, i.e., freecholesterol.

As used herein, “cholesterol-containing particle” includes, without limitation, both naturally occurring and recombinant low density lipoproteins, as well as synthetic cholesterol-containing particles. Cholesterol-containing particles must be able to enter a cell and thereby serve as a vehicle for the importation of cholesterol into the cell.

As used herein, “cholesterol efflux” shall mean the movement of cholesterol from a cell to the cell's exterior, and/or any biochemical step constituting part of such movement. In one embodiment, cholesterol is moved from a cell to a cholesterol acceptor which then transports the cholesterol out of the cell.

As used herein, a “cholesterol-loaded” cell shall mean a cell having a level of cholesterol higher than normal for that cell type. For example, if a human macrophage has a cholesterol level of X, and a human macrophage in question has a cholesterol level of 2X, the human macrophage in question is considered “cholesterol-loaded.” A higher than normal cholesterol level can be any level higher than normal including, for example, 1%, 2%, 5%, 10%, 20%, 50%, and 100% higher than normal. In one embodiment, free cholesterol-loaded cells are formed in culture by human intervention. This is accomplished, for example, by contacting the cells in culture with a cholesterol-containing particle, such as an acyl low density lipoprotein, under conditions where ACAT is inhibited. If ACAT is not inhibited, then the cells become loaded primarily with cholesteryl esters instead of free cholesterol.

As used herein, “HDL” shall mean “high-density lipoprotein.” HDL is the main extracellular acceptor of cholesterol, and transports cholesterol to the liver for excretion.

“Niemann-Pick C molecule”, abbreviated herein as “NPC”, includes, without limitation, type I and type II molecules. These NPC molecules play an important role in intracellular cholesterol trafficking, particularly in the exit of cholesterol from late endosomes or lysosomes.

As used herein, “U18666A” shall mean the amphipathic amine 2β-(2-diethlaminoethoxy)-androstenone.

Embodiments of the Invention

This invention provides a first method for determining whether an agent increases ABCA1-dependent cholesterol efflux from a cell which comprises (a) contacting a free cholesterol-loaded cell with the agent in the presence of a cholesterol acceptor which binds to cholesterol effluxed from a cell via an ABCA1-dependent pathway, (b) quantitatively determining the efflux of cholesterol from the cell, and (c) comparing the efflux so determined with a known standard, thereby determining whether the agent increases cholesterol efflux from the cell.

The determination of an “increase” in free cholesterol efflux is made by comparison to a known standard. For example, cholesterol efflux from a cell in the absence of the agent but otherwise under conditions identical to those used in the presence of the agent, is one possible standard. In this example, an increase in cholesterol efflux in the presence of the agent relative to that in the absence of the agent indicates that the agent increases cholesterol efflux. The efflux is characterized as ABCA1-dependent by virtue of the cholesterol acceptor used. For example, ABCA1 binds with high affinity to apoAI, but not to HDL₂. Cholesterol efflux to apoAI is therefore characterized as ABCA1-dependent.

In one embodiment, the cholesterol acceptor is selected from the group consisting of apolipoprotein A-I, apolipoprotein A-II, apolipoprotein A-IV, apolipoprotein E, a recombinant apolipoprotein and a synthetic apolipoprotein. In the preferred embodiment, the acceptor is apolipoprotein A-I.

In one embodiment, the free cholesterol-loaded cell is produced by contacting a cell with a cholesterol-containing particle, whereby the particle enters the cell, and contacting the cell with an acyl-CoA-cholesterol acyltransferase inhibitor so as to inhibit the activity of acyl-CoA-cholesterol acyltransferase in the cell. These steps may be performed concurrently or in any other order. For example, the cell may be contacted with the inhibitor either prior to or after the cell is contacted with a cholesterol-containing particle. In the preferred embodiment, the cholesterol-containing particle is an acetyl low density lipoprotein.

In another embodiment, the free cholesterol-loaded cell comprises detectably labeled cholesterol and quantitatively determining the efflux of cholesterol from the cell comprises quantitatively determining the efflux from the cell of the detectably labeled cholesterol. In one embodiment, the detectable label is a radioisotope, preferably tritium or carbon-14.

This invention also provides a second method for increasing cholesterol efflux from a cell comprising contacting the cell with an agent which increases ABCA1-dependent cholesterol efflux from a cell.

This invention further provides a third method for decreasing the amount of cholesterol in a cell comprising contacting the cell with an agent which increases ABCA1-dependent cholesterol efflux from the cell.

In one embodiment of any of the instant methods, the cell is selected from the group consisting of a macrophage, a hepatic cell and a smooth muscle cell. In a preferred embodiment, the cell is a macrophage. In another embodiment, the cell is a human cell.

This invention also provides a fourth method for increasing the likelihood that a cholesterol-loaded macrophage will survive comprising contacting the macrophage with an agent which increases ABCA1-dependent cholesterol efflux from a macrophage, thereby increasing the likelihood that the macrophage will survive.

This invention further provides a fifth method for decreasing the likelihood that a cholesterol-loaded macrophage will contribute to the progression of atherosclerosis in a subject comprising contacting the macrophage with an agent which increases ABCA1-dependent cholesterol efflux from a macrophage, thereby decreasing the likelihood that the macrophage will contribute to the progression of atherosclerosis in the subject. In a preferred embodiment the subject is a human. In a further embodiment, the agent is admixed with a pharmaceutically acceptable carrier.

This invention also provides a sixth method for treating a subject afflicted with atherosclerosis comprising administering to the subject a therapeutically effective amount of an agent which increases ABCA1-dependent cholesterol efflux from a cell, thereby treating the subject. In a preferred embodiment, the subject is a human. In one embodiment, the therapeutically effective amount of the agent is less than about 3.75 mg of agent per kg of the subject's body weight. In a preferred embodiment, the therapeutically effective amount of the agent is about 0.75 mg of agent per kg of the subject's body weight. In a further embodiment, the agent is admixed with a pharmaceutically acceptable carrier.

In one embodiment of any of the fourth through sixth methods, the agent is U18666A or a pharmaceutically acceptable salt thereof. In this embodiment, the agent, when contacted with the cell, can be, for example, at a concentration of from about 30 nM to about 120 nM, and preferably, about 70 nM.

In another embodiment of any of the fourth through sixth methods, the agent is imipramine or a pharmaceutically acceptable salt thereof. In this embodiment, the agent, when contacted with the cell, can be for example, at a concentration of from about 2 μM to about 20 μM, and preferably, about 8 μM. Pharmaceutically acceptable salts are well known in the art and include, without limitation, salts of Na⁺, K⁺, Mg⁺⁺ and various amines (Int'l. J. Pharm. (1986) 33:201-217).

In one embodiment of any of the fourth through sixth methods, the agent is an inhibitor of an intracellular cholesterol trafficking pathway. In another embodiment, the intracellular cholesterol trafficking pathway is mediated by a Niemann-Pick C molecule, lysobisphosphatidic acid, and/or lysosomal sphingomyelinase.

In another embodiment of any of the fourth through sixth methods, the agent protects the ABCA1 protein from degradation. Degradation of the ABCA1 protein may be induced, for example, by an accumulation of intracellular free cholesterol, or by an NPC1-dependent mechanism. In yet another embodiment, the agent protects ABCA1 from cell death or apoptosis.

Finally, this invention provides an article of manufacture comprising packaging material and a pharmaceutical agent, wherein the pharmaceutical agent increases ABCA1-dependent cholesterol efflux from a cell and wherein the packaging material comprises a label indicating that the pharmaceutical agent is intended for use in treating a subject afflicted with atherosclerosis. In the preferred embodiment, the subject is a human. Also in the preferred embodiment, the cell is a macrophage cell.

In one embodiment of the article of manufacture, the agent is an inhibitor of an intracellular cholesterol trafficking pathway mediated by a Niemann-Pick C molecule, lysobisphosphatidic acid, and/or lysosomal sphingomyelinase.

In another embodiment of the article of manufacture, the agent is U18666A or a pharmaceutically acceptable salt thereof. In a further embodiment, the agent is imipramine or a pharmaceutically acceptable salt thereof.

This invention is illustrated in the Experimental Details section which follows. This section is set forth to aid in an understanding of the invention but is not intended to, and should not be construed to, limit in any way the invention as set forth in the claims which follow thereafter.

Experimental Details

Synopsis

The accumulation of large amounts of free cholesterol eventually leads to macrophage death, resulting in lesional necrosis. Hence, the free cholesterol-loaded macrophage is likely to be a critical turning point in the progression of atherosclerosis. In support of this hypothesis, lesional necrosis is a precipitating factor in plaque erosion and rupture, which in turn leads directly to acute thrombosis and acute vascular occlusion. Thus, the prevention of free cholesterol-induced macrophage death is a novel and important therapeutic strategy for the prevention of these fatal steps in atherosclerotic plaque progression.

The results described herein demonstrate that free cholesterol-loading of macrophage cells causes a reduction in ABCA1-dependent efflux activity accompanied by a proteosome-dependent decrease in ABCA1 protein levels. Further disclosed is the surprising result that low concentrations of amphipathic amines such as imipramine and 3β-(2-diethylaminoethoxy)-androstenone (U-18666A) markedly enhance ABCA-1 mediated-cholesterol efflux in free cholesterol-loaded cells. This evidence suggests that this protective effect is due to a partial inhibition of NPC1-dependent intracellular cholesterol trafficking.

Methods

Materials

Tissue culture media were from Life Technologies, Inc., and fetal bovine serum (FBS) was from Hyclone Laboratories (Logan, Utah). Tritium-labeled cholesterol and choline were from Perkin-Elmer Life Sciences, Inc. (Boston, Mass.). Concanavalin A, ALLN, methyl-β-cyclodextrin, and imipramine were from Sigma. Compound 58035 (3-[decyldimethylsilyl]-N-[2- (4-methylphenyl)-1-phenylethyl] propanamide, may be obtained from Sandoz, Inc. (East Hanover, N.J.); a 10 mg/ml stock solution was prepared in dimethyl sulfoxide, and the final dimethyl sulfoxide concentration in both treated and control cells was 0.05%. Glyburide, sodium orthovanadate, lactacystin, and cycloheximide were from Calbiochem. U18666A was from Biomol Research Lab, Inc. Apolipoprotein A-I (apoA-I) was from Biodesign International (Saco, Me.), and rabbit anti-ABCA1 serum was from Novus (Littleton, Colo.) Anti-β-actin, HRP-conjugated goat anti-rabbit IgG, and goat anti-mouse IgG were from Bio-Rad. LDL (d, 1.020-1.063 g/ml) and HDL₂ (d, 1.063-1.125 g/ml) from fresh human plasma were isolated by preparative ultracentrifugation. Acetyl-LDL was prepared by reaction with acetic anhydride and labeled with ³H-CE.

Harvesting and Culturing Mouse Peritoneal Macrophages

The mice used in this study were wild-type C57BL6/J and BALB/c; C57BL6/J apoE KO; C57BL6/J apoE KO Nctr-npcl^{N} heterozygous; and BALB/cNctr-npcl^{N} heterozygous mice. The C57 heterozygous NPC1 apoE KO mice were produced by crossing BALB/cNctr-npcl^{N} mice (stock number 003092; Jackson Laboratory, Bar Harbor, Me.) onto C57B6/J apoE KO background for five generations. Six-ten week-old mice were injected with 0.5 ml PBS containing 40 μg of concanavalin A intraperitoneally, and the macrophages were harvested three days later by peritoneal lavage. The harvested cells were plated in cell-culture plates in Dulbecco's modified Eagle's medium (DMEM) supplemented with 10% fetal bovine serum (FBS) and 20% L-cell conditioned medium. The medium was replaced every 24 hours until the macrophages were confluent, at which point they were incubated with 50-100 μg/ml acetyl-LDL in DMEM containing 0.2% BSA with or without 10 ug/ml 58035 and/or other inhibitors.

³H-Cholesterol Efflux Assay

Acetyl-LDL (800 μg) was incubated with 10 μCi ³H-cholesterol for 30 min at 37° C., followed by addition of 8 ml of DMEM, 0.2% BSA. The macrophages were incubated with this medium for 5 h, washed 3 times with PBS, and then incubated with DMEM, 0.2% BSA for 15 min at 37° C. After washing with PBS, the macrophages were incubated with DMEM, 0.2% BSA, containing either 15 μg/ml apoAI or 20 μg/ml HDL₂. At the indicated time points, 100 μl of media was removed and centrifuged for 5 min at 14,000 rpm to pellet cellular debris. The radioactivity in this fraction of media was quantitated by liquid scintillation counting. After the last time point, the remainder of the media was removed, and the cells were dissolved in 0.5 ml of 0.1 N NaOH containing 0.5% sodium dodecylsulfate (5 h at room temperature). A 100 μl-aliquot of the cell lysate was counted, and the percent efflux was calculated as (media cpm)÷(cell+media cpm)×100. Total protein in the cell lysate was determined using the Bio-Rad DC protein assay kit. Note that there was no statistical difference in cellular cpm or protein between cholesteryl ester- and free cholesterol-loaded macrophages.

³H-Phospholipid Efflux Assay

Macrophages were labeled with ³H-choline (5 μCi/ml) in DMEM, 10% FBS, for 24 h. After washing three times with PBS, the macrophages were incubated with 100 μg/ml acetyl-LDL±58035 in DMEM, 0.2% BSA, for 5 h. The cells were then incubated with 15 μg/ml apoA-I in DMEM, 0.2% BSA, for the indicated time periods. ³H-choline-containing phospholipids in aliquots of the medium were extracted in chloroform:methanol (2:1, v/v), and those remaining in the cells in hexane:isopropyl alcohol (3:2, v/v). The radioactivity was measured by scintillation counting.

Whole-Cell Cholesterol Esterification Assay

Macrophages were incubated in DMEM, 0.2% BSA, containing 0.1 mM ¹⁴C-oleate complexed with albumin and 3 μg/ml acetyl-LDL. At the indicated time points, the cells were washed two times with cold PBS, and the cell monolayers were extracted twice with 0.5 ml of hexane/isopropyl alcohol (3:2, v/v) for 30 min at room temperature. Whole-cell cholesterol esterification activity was assayed by determining the cellular content of cholesteryl ¹⁴C-oleate by thin-layer chromatography. The cell monolayers were dissolved in 1 ml of 0.1 N NaOH, and aliquots were assayed for protein by the Lowry method.

Biotinylation of Cell-Surface Proteins

Macrophage monolayers in 35-mm dishes were washed with ice-cold PBS 3 times and then incubated with ice-cold PBS containing 0.5 mg/ml NHS-SS-biotin (Pierce) for 30 minutes at 4° C. After washing 5 times with ice-cold PBS containing 20 mM Tris-HCl, pH 8.0, the cells were scraped into PBS and pelleted by centrifugation. The pelleted macrophages were lysed in 50 μl RIPA buffer (0.5% sodium deoxycholate, 0.1% SDS, 1% Triton X-100, 20 mM Tris, 150 mM NaCl, and 5 mM EDTA, pH 8) containing 1 mM PMSF. Ten μl of the lysate were subjected directly to 4-20% gradient SDS-polyacrylamide gel electrophoresis (SDS-PAGE) for determination of total ABCA1. The rest of the cell lysate was affinity-purified to isolate biotinylated proteins. Briefly, the cell lysates were diluted to 150 μl in RIPA buffer and incubated with 50 μl immobilized streptavidin agarose (Pierce), which was pre-washed three times with RIPA buffer at 0C for 2 h with gentle shaking. The agarose was pelleted by centrifugation using a microcentrifuge at 5,000 rpm for 2 min; the pellet was resuspended in 1 ml RIPA buffer, and the process was repeated 5 times. The agarose was resuspended 30 μl SDS-PAGE loading buffer containing 330 mM β-mercaptoethanol at 37° C. for 15 min and subjected to SDS-PAGE as above. ABCA1 and β1-integrin were detected by Western blot using anti-ABCA1 and anti-β1-integrin anti-sera. The blots were reprobed with anti-β-actin antibody, which detected no actin signal, thus verifying that no cytosolic protein was biotinylated by the procedure.

Western Blot Analysis

Peritoneal macrophages were lysed in RIPA buffer containing 1 mM PMSF. Nuclei were removed by centrifugation at 3000×g for 10 min at 4° C. Protein in the supernatants (15-30 μg of protein) was separated by electrophoresis on 4-20% gradient SDS-PAGE and electrotransferred to a 0.22-μm nitrocellulose membrane using a Bio-Rad mini transfer tank (Bio-Rad). For Western blot detection of ABCA1, anti-ABCA1 antiserum was used. Signals were detected using HRP-conjugated secondary anti-bodies (Bio-Rad) and ECL (Amersham Pharmacia Biotech). The membranes were reprobed with anti-β-actin monoclonal antibody or anti-β1-integrin anti-serum for the proper internal controls. The relative intensities of the bands were determined by densitometry.

Real-Time Quantitative RT-PCR

Monolayers of macrophages in 22-mm dishes were incubated for 5 h with 100 μg/ml acetyl-LDL in the absence or presence of 10 μg/ml 58035. After washing with cold PBS, the cells were lysed with 1 ml Trizol reagent to isolate total RNA. Five μg total RNA was reversed transcribed using BRL Superscript II and polyT as the primer, and PCR was conducted using 62.5 ng cDNA in the Mx4000TM Multiplex Quantitative PRC system from Stratagene. The primers for the ABCA1 gene were 5′-cctcagccatgacctgccttgtag-3′ and 5′-ccgaggaagacgtggacaccttc-3′. To control for input cDNA, a β-actin primer/probe set from PE Biosystems was used. The PCR products were checked by agarose gel electrophoresis to make sure a single PCR product was obtained. A standard curve was obtained by plotting the cycle threshold versus the log of input cDNA, which was obtained from CE-loaded mouse peritoneal macrophages. Both the β-actin and ABCA-I standard curves were linear between 31.25 and 250 ng CDNA (r²=0.99 for both). The PCR reactions were set up using SYBR-Green PCR Core Reagents from Applied Biosystems. The PCR was initiated at 95° C. for 10 min, followed by 45 cycles consisting of 95° C. for 0.5 min, 56° C. for 1.5 min, and 72° C. for 1.4 min. After obtaining real time fluorescence measurements, cycle threshold values were determined. Using the standard curves in the linear range (i.e., exponential amplification phase), the quantities of ABCA-I and β-actin mRNAs were calculated. The final data are expressed as the ratio of ABCA1:β-actin mRNA.

In Vivo Efficacy of U18666A

LDL receptor knockout mice were fed a diet containing cholesterol and saturated fat for 12 weeks in the absence or presence of 0.75 mg/kg/d U18666A (10 mice per group).

Statistics

Results are given as means±S.E.M. (n=3); absent error bars in the figures signify S.E.M. values smaller than the graphic symbols. For the quantitative PCR measurements, triplicate values were obtained, and there was <1% variation among these values.

Results

Free Cholesterol Loading of Macrophages Leads to the Dysfunction of the ABCA1 Cholesterol Efflux Pathway

In order to mimic the pathology of the cholesterol-loaded macrophage that occurs in atherosclerotic lesions, an assay was developed wherein cultured peritoneal macrophages are induced to accumulate excess cholesterol, either predominantly in the form of cholesteryl esters or in the form of free cholesterol. The relative effect of cholesteryl ester loading versus free cholesterol loading on cholesterol efflux from the cells was then determined. ApoA-I was used as an ABCA1-specific cholesterol acceptor protein in order to differentiate ABCA1-dependent from ABCA1-independent efflux in this assay.

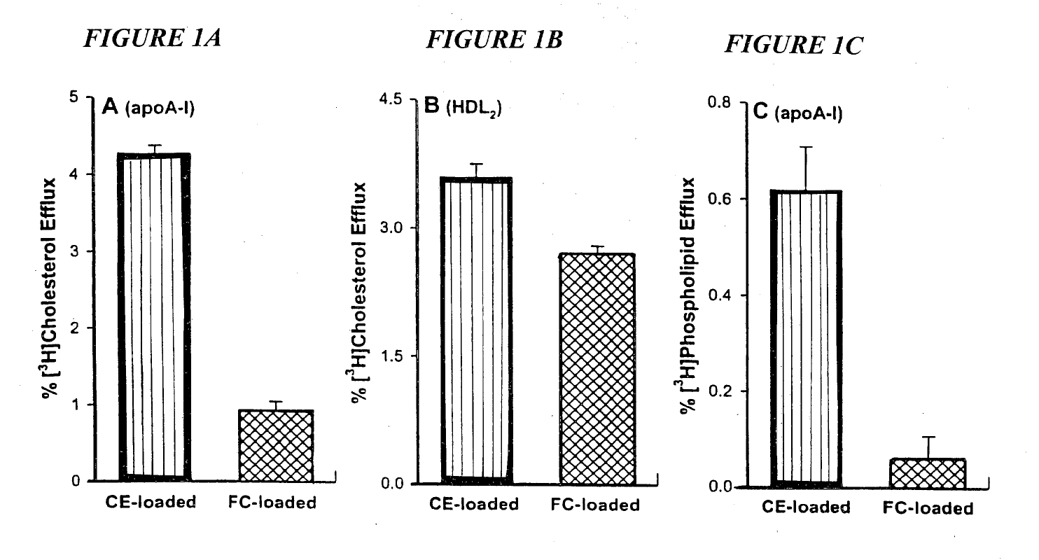

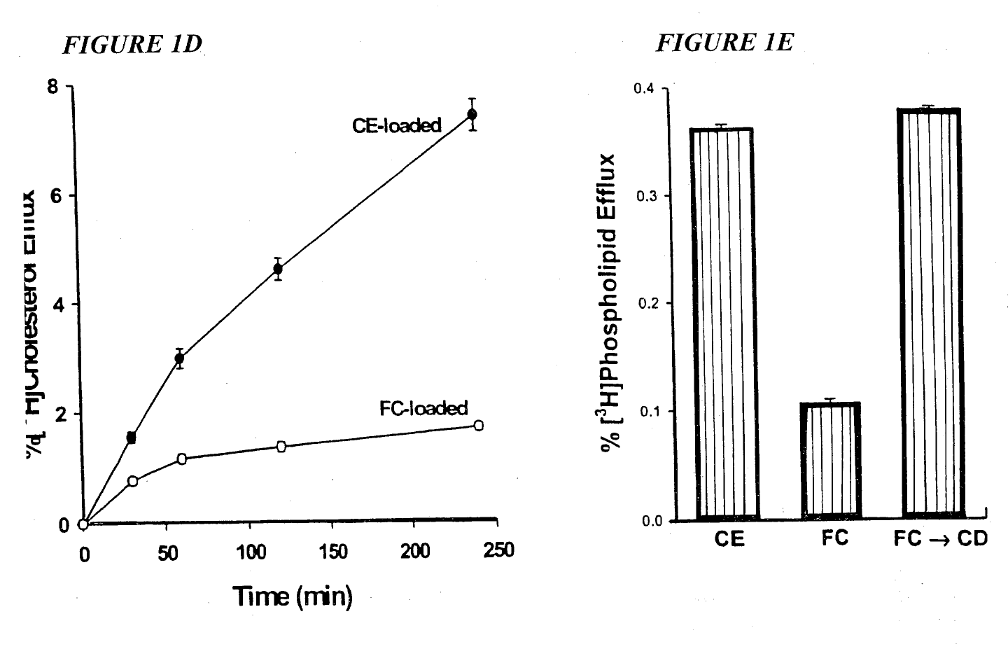

Mouse peritoneal macrophages were incubated with tritiated cholesterol-labeled acetyl-LDL either alone, to effect predominantly cholesteryl ester loading, or in the presence of the ACAT inhibitor, 59035, for free cholesterol loading. Cholesterol efflux was measured in the presence of either apoA-I or HDL₂, which does not bind ABCA1 and therefore serves as a measure of efflux through ABCA1-independent pathways. As shown in FIG. 1, free cholesterol-loaded cells demonstrated a marked reduction in cholesterol efflux to apoA-I (FIG. 1A), while only modestly affecting efflux to HDL₂ (FIG. 1B). Furthermore, as shown in FIG. 1D, the free cholesterol-induced defect in efflux to ApoA-I was manifest within the first hour following cholesterol loading of the cells. These results indicate both that ABCA1 transporter activity was particularly sensitive to impairment by excessive intracellular free cholesterol and that its impairment is an early event following free cholesterol accumulation.

In order to examine the relative activity of ABCA1 in the cholesterol-loaded cells, phosphatidylcholine efflux to apoA-I was measured in both free- and cholesteryl ester-loaded macrophages. This assay is based on a model in which ABCA1-mediated cholesterol efflux is divided into two sequential steps, (i) phospholipid efflux to lipidfree apoA-I, and (ii) cholesterol efflux to these apoA-Iphospholipid complexes. Relying on this model, a defect in phospholipid efflux indicates reduced ABCAL transporter activity.

As demonstrated in FIG. 1C, free cholesterol-loaded cells exhibited substantially reduced phosphatidylcholine efflux compared with that of cholesteryl ester-loaded cells. Furthermore, as shown in FIG. 1E, this defect was reversed by treatment of the cells with methyl-β-cyclodextrin, which removes free cholesterol. These results demonstrate that ABCA1 transporter activity is compromised in free cholesterol-loaded cells and that this defect is largely due to the free cholesterol itself.

FC-Loading of Macrophages Leads to a Decrease in ABCA1 Protein but not in ABCA1 mRNA

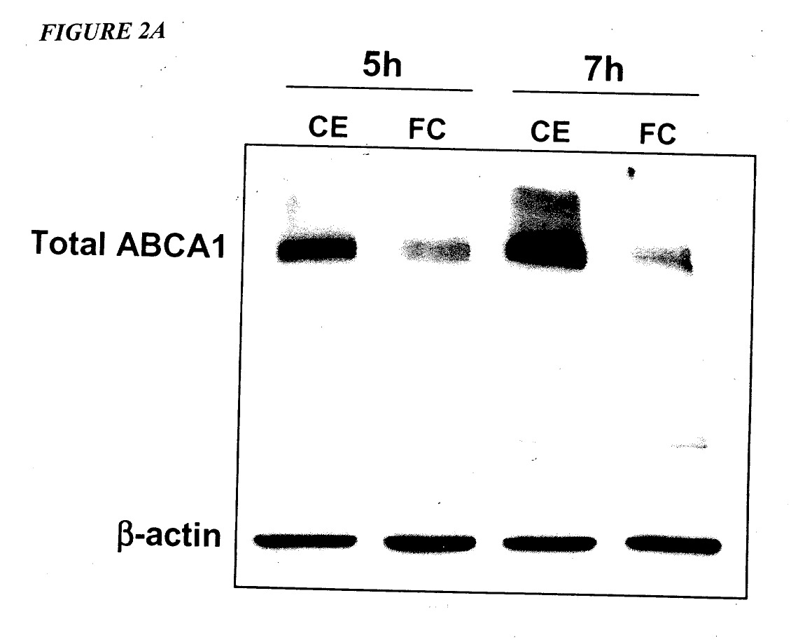

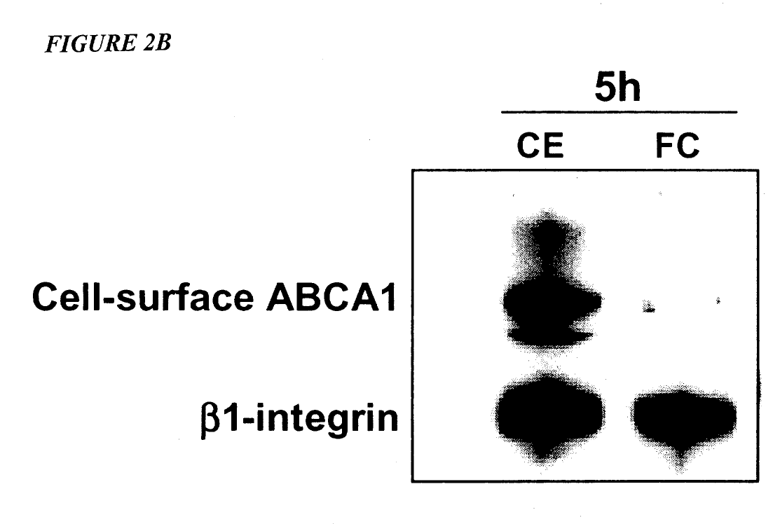

In order to determine whether the decrease in ABCAL transporter activity in free cholesterol-loaded macrophages correlated with lower protein levels, lysates from cells that were cholesterol loaded for either 5 or 7 hours were analyzed for ABCA1 protein expression using standard western immunoblotting techniques. As shown in FIG. 2A, total ABCA1 protein was substantially decreased in free cholesterol-loaded cells compared with cholesteryl ester-loaded cells at both the 5 and 7 hour time points. Normalized to β-actin, the data demonstrated a 75% decrease in ABCA1 expression at 5 h and a greater than 90% decrease at 7 h. In contrast, the cholesteryl ester-loaded cells showed a 2.4-fold increase in ABCA1 protein expression between 5 and 7 h, consistent with the induction of ABCA1 expression previously reported in response to sterol loading. As shown in FIG. 2B, the decreased expression of ABCA1 in free cholesterol-loaded cells was even more pronounced in the membrane fraction.

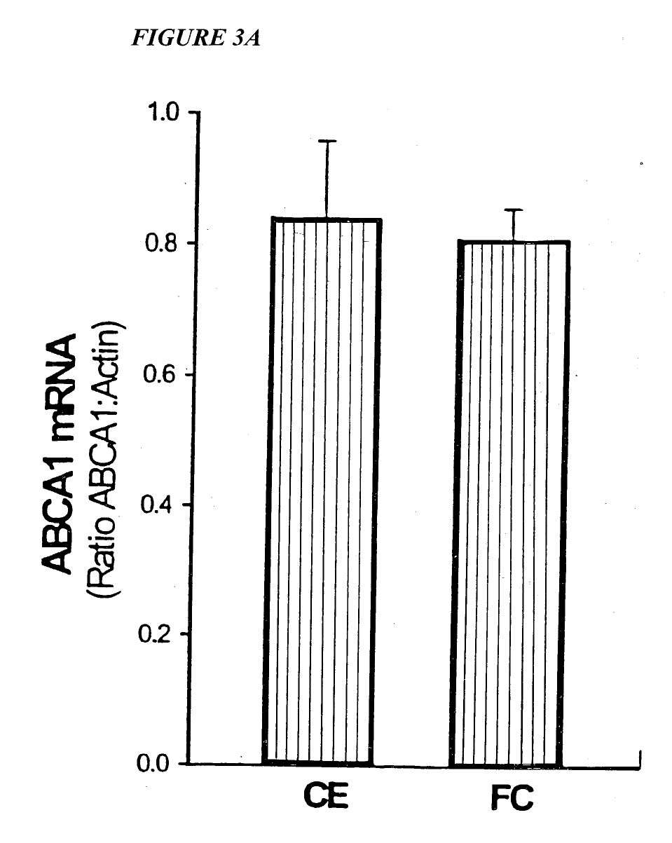

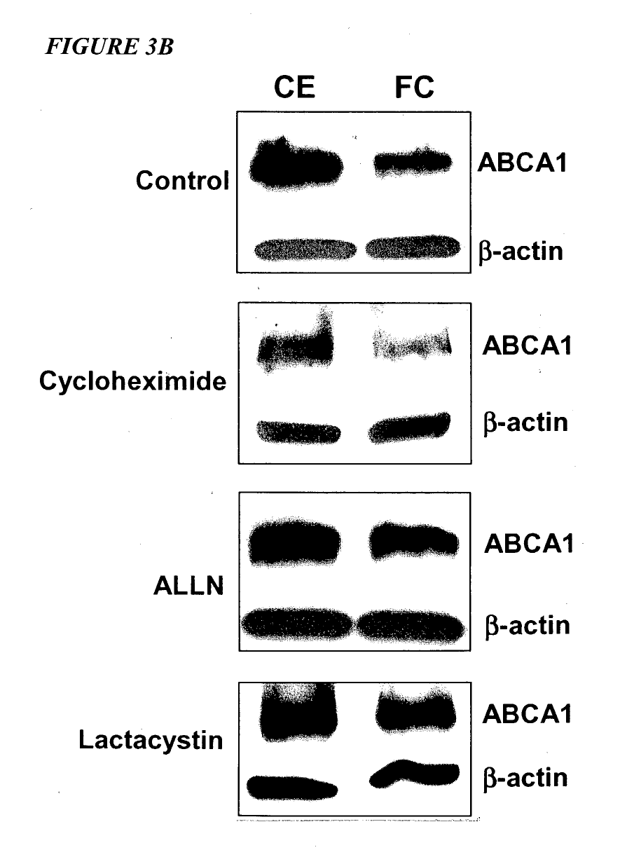

These results indicate that the expression of ABCA1, particularly that of the membrane-associated protein, was substantially diminished in free cholesterol-loaded cells. As shown in FIG. 3A, this decreased expression of the protein did not correlate with a reduction in ABCA1 mRNA levels. It was therefore determined whether there was reduced translation of the ABCA1 mRNA in free cholesterol-loaded cells. To do this, cycloheximide-treated cells were cholesterol-loaded and examined for ABCA1 protein expression. As shown in FIG. 3B (top and middle blot), the decrease in ABCA1 protein observed in free cholesterol-loaded cells was insensitive to cycloheximide. Together with the mRNA data, these results indicate that a post-translational mechanism is likely to be responsible for the observed decrease in ABCA1 protein levels. Consistent with this, both ALLN, an inhibitor of cysteine proteases and proteasomal degradation, and lactacystin, a specific inhibitor of proteasomal degradation, blocked the decrease in ABCA1 in free cholesterol-loaded macrophages. Inhibitors specific for the cysteine protease calpain, calpeptin (40 μM) and PD150606 (25 μM), did not affect the decrease in ABCA1 in FC-loaded macrophages (data not shown).

In summary, these results demonstrate that free cholesterol-loading of macrophages results in a substantial decrease in ABCA1 protein expression, most likely through increased proteasome-dependent degradation.

Studies with Heterozygous NPC1 Mutant Macrophages

These results thus far indicate that free cholesterol-loading leads to defective ABCA1-mediated cholesterol efflux and increased turnover of the ABCA1 protein. Since ABCA1 functions as a transporter, it was determined whether free cholesterol-loading is also associated with defects in intracellular cholesterol transport using macrophage cells from mice carrying a heterozygous deletion in the gene for NPC1.

NPC1, the protein defective in type I Niemann-Pick C disease, is required for the normal trafficking of cholesterol out of late endosomal and/or lysosomal compartments. In free cholesterol-loaded macrophages, cholesterol accumulates in perinuclear organelles, presumably late endosomes or lysosomes, and also traffics to peripheral sites, such as the plasma membrane and endoplasmic reticulum. It was previously shown that cholesterol efflux via both ABCA1-dependent and independent pathways is severely disrupted in macrophages from homozygous NPC1 knockout mice, presumably because cholesterol transport from late endosomes and/or lysosomes to the ABCA1 efflux pathway in the plasma membrane is defective.

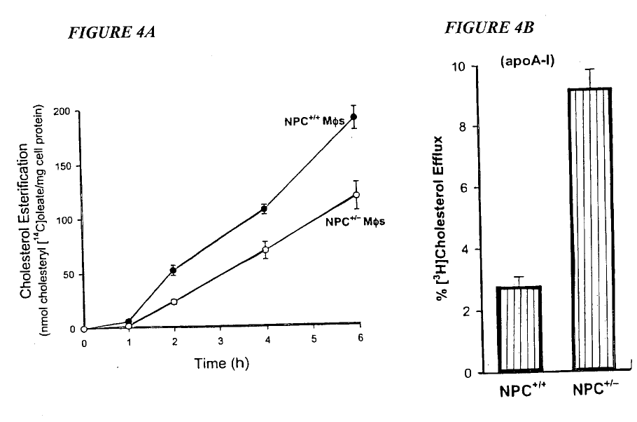

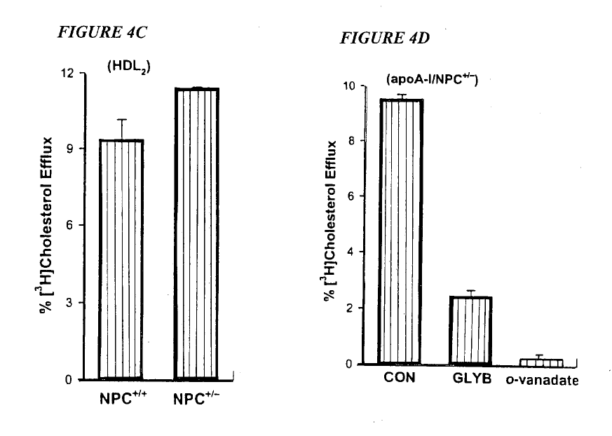

NPC1 heterozygous macrophage cells provide a system in which cholesterol trafficking to the plasma membrane remains mostly intact while trafficking to other intracellular peripheral sites is severely compromised. It was demonstrated previously that NPC1 heterozygotes exhibit only a slight defect in cholesterol trafficking to the plasma membrane (about a 10-15% decrease compared with wild-type cells). However, as shown in FIG. 4A, trafficking to the endoplasmic reticulum was decreased by as much as 50% in these cells, consistent with the requirement for NPC1 in cholesterol transport from late endosomes and/or lysosomes.

To examine the effects of cholesterol-loading in this system, cholesterol-loaded wild-type (NPC^{+/+}) and heterozygous mutant (NPC^{+/-}) macrophages were assayed for efflux to apoA-I and HDL₂ as described previously. Importantly, there was no significant difference in cholesterol loading between the two genotypes. As shown in FIG. 4B, cholesterol efflux to apoA-I was markedly increased in the NPC^{+/-} macrophages compared with NPC^{+/+} macrophages. This efflux was effectively blocked by two inhibitors of the ABCA1 efflux pathway, glyburide and ortho-vanadate (FIG. 4D), demonstrating that the increased efflux was ABCA1-dependent. As expected from the previous data, ABCA1-independent efflux to HDL₂ was already relatively high in NPC^{+/+} free cholesterol-loaded macrophages, and it was increased only slightly by the heterozygous NPC mutation (FIG. 4C). Thus, NPC^{+/-} macrophages were protected from the free cholesterol-induced defect in the ABCA1-dependent efflux pathway.

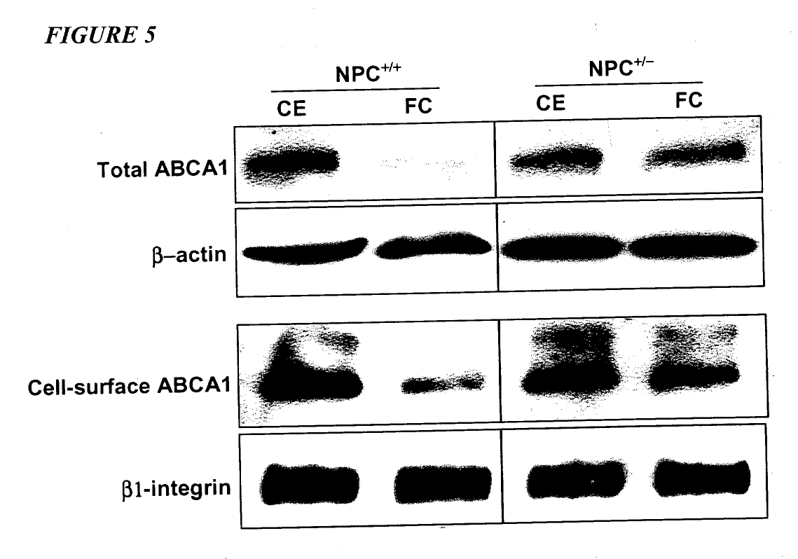

It was next determined whether rescue from the defect in cholesterol efflux was accompanied by an increase in ABCA1 protein expression in the NPC^{+/-} macrophages. Consistent with earlier results, there was an approximately 95% decease in total ABCA1 protein and an approximately 80% decrease in cell-surface ABCA1 protein in free cholesterol-loaded NPC^{+/+} macrophages (FIG. 5). Strikingly, NPC^{+/-}0 macrophages exhibited only about a 5% decease in total ABCA1 and a 25% decrease in cell-surface ABCA1.

Together, these data indicate that free cholesterol-loading induces degradation of ABCA1 and that the resulting defect in cholesterol efflux to apoA-I requires intact trafficking of free cholesterol to a peripheral cellular site. Furthermore, these data indicate that a partial inhibition of intracellular cholesterol trafficking offers a dramatic protective effect against free cholesterol-induced defects in ABCAL mediated efflux.

Studies with Low-Dose Amphipathic Amines

In order to test the idea that partial disruption of cholesterol trafficking offers a protective effect to ABCA1 in free cholesterol-loaded cells, the ability of certain types of amphipathic amines, such as 2β-(2diethlaminoethoxy)-androstenone (U18666A) and imipramine, to mimic the NPC mutant phenotype was exploited.

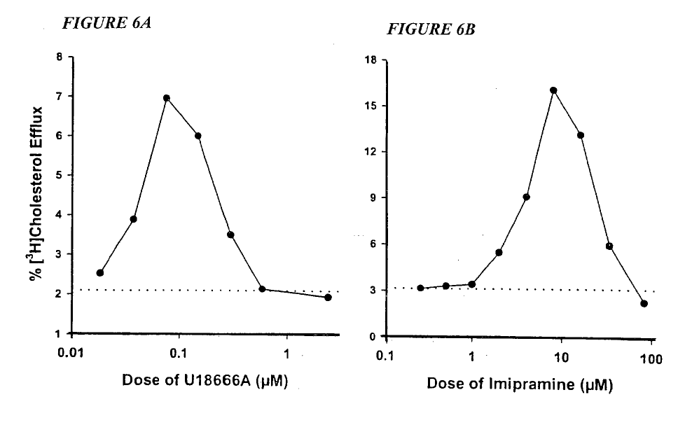

Efflux from free cholesterol-loaded macrophage cells was measured as described previously in the absence or presence of either U18666A or imipramine, as indicated in FIG. 6. Notably, both compounds exhibited a marked ability to induce cholesterol efflux to ApoA-1. Peak efflux was observed at 70 nM for U18666A (FIG. 6A), which was almost 100-fold less than the peak concentration for imipramine (FIG. 6B). At concentrations greater than 100 nM, U18666A gradually inhibited efflux, presumably due to a severe blockage of cholesterol trafficking to the plasma membrane. A similar biphasic profile was observed with imipramine. Importantly, 70 nM U18666A decreased cholesterol trafficking to the endoplasmic reticulum by about 90% decrease while trafficking to the plasma membrane was reduced by only about 10% (data not shown).

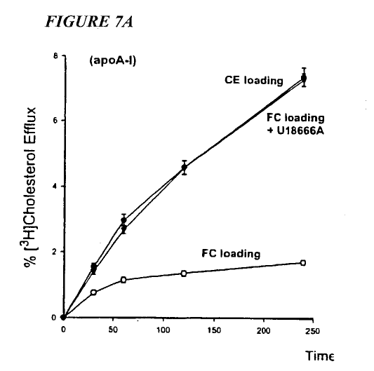

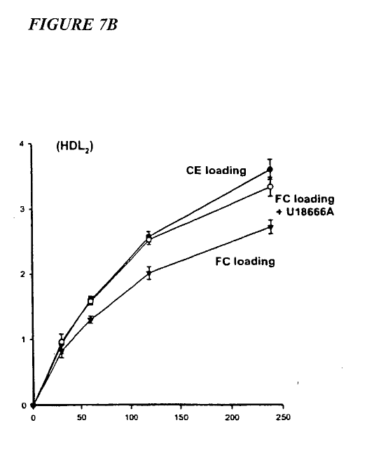

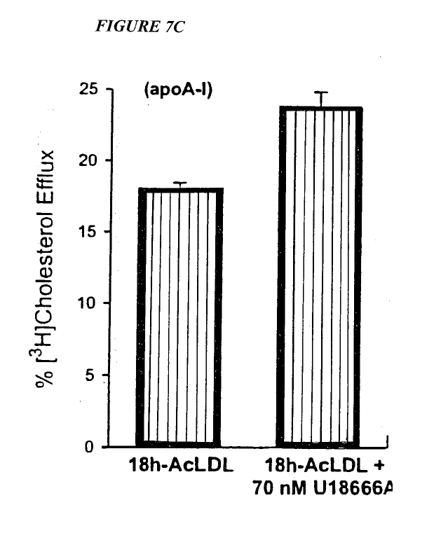

These results suggest that optimal low doses of inhibitors such as U18666A and imipramine restored ABCA1-dependent efflux in free-cholesterol loaded macrophages. As shown in FIG. 7A, this effect was sufficient to restore efflux to the levels observed in cholesteryl ester-loaded cells. In addition, while U18666A improved both ABCA1-dependent and independent efflux from free cholesterol-loaded cells, the net effect was substantially greater for ABCA1-dependent efflux (compare FIGS. 7A and 7B).

Notably, as shown in FIG. 7C, 70 nM U18666A also increased cholesterol efflux to apoA-I by about 30% in macrophages incubated for a prolonged period with acetyl-LDL without an ACAT inhibitor. These data raise the possibility that the amount of free cholesterol that naturally accumulates under these conditions may be enough to cause modest dysfunction of the ABCA1 cholesterol efflux pathway.

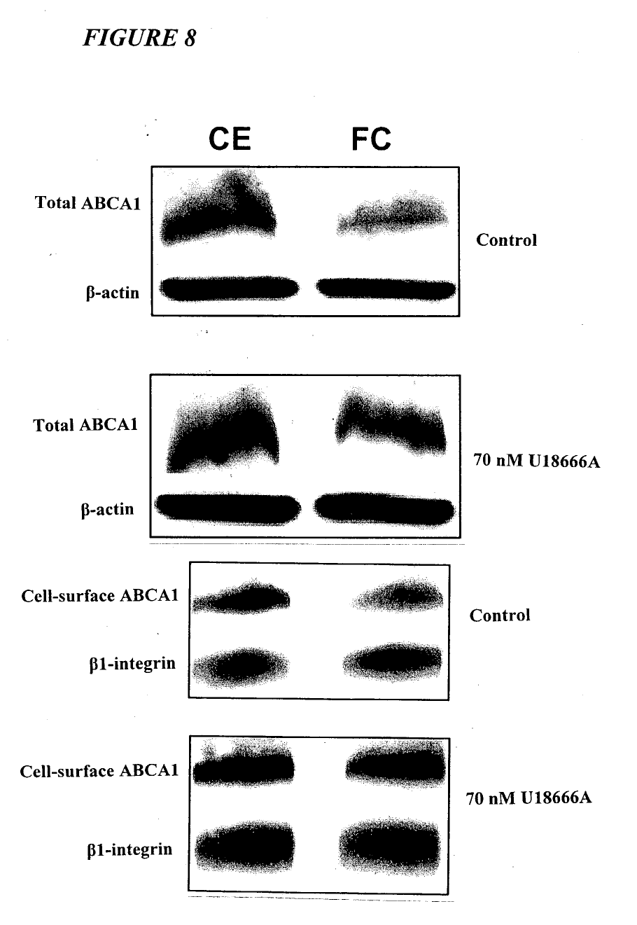

Consistent with its ability to restore ABCA1-mediated cholesterol efflux, U18666A also protected from the ABCA1 protein loss observed in free cholesterol-loaded cells (FIG. 8, top panel). This protective effect was particularly striking in the case of cell-surface ABCA1 protein, which decreased by only about 15% in U18666A-treated cells, compared with 60% in untreated cells (FIG. 8, bottom panel).

In Vivo Efficacy of U18666A in a Mouse Model of Atherosclerosis

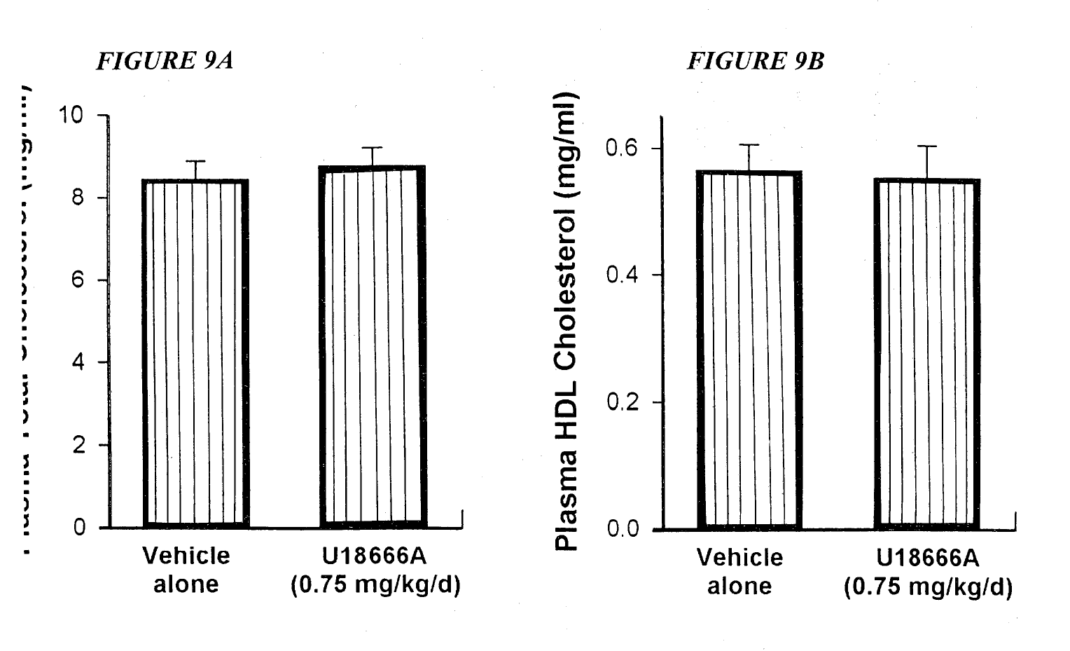

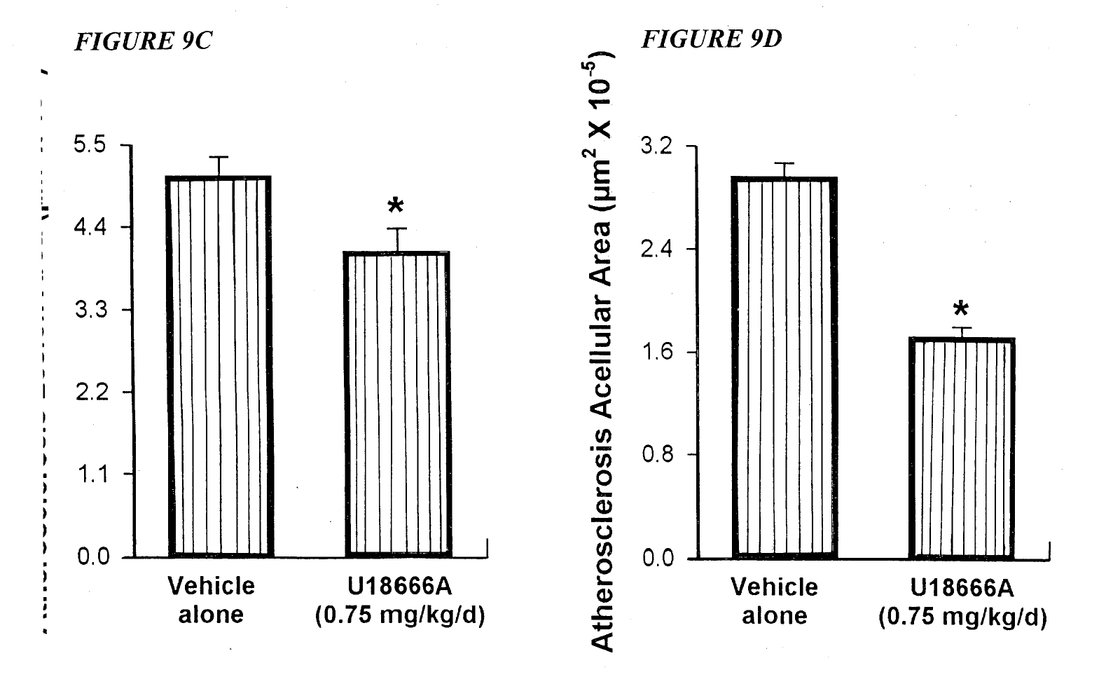

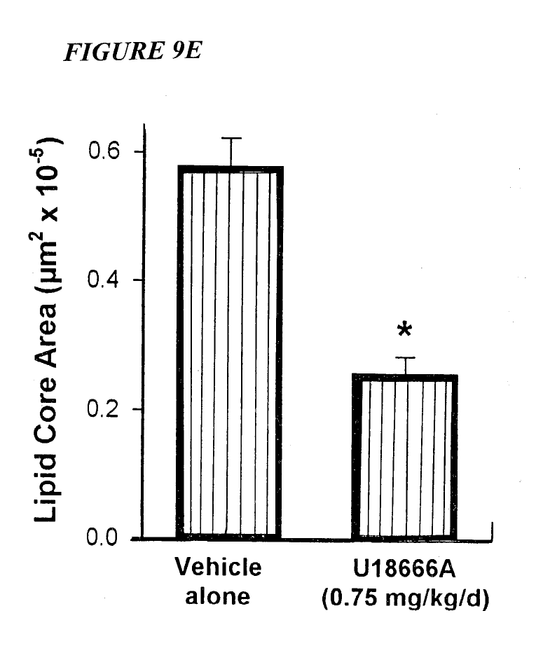

In order to determine if the ability of U18666A to maintain ABCA1-dependent efflux in free cholesterol-loaded cells translates into a protective effect against atheroslerosis in vivo, the effect of low-dose U18666A on lesion development in the LDL receptor knockout mouse model was examined. LDL receptor knockout mice were fed a diet containing cholesterol and saturated fat for 12 weeks in the absence or presence of 0.75 mg/kg/d U18666A (10 mice per group). As shown in FIG. 9, the plasma levels of both total cholesterol (FIG. 9A) and HDL (FIG. 9B) cholesterol are similar in the U18666A-treated group compared to those receiving vehicle alone. However, the U18666A treatment group exhibited a marked reduction in atherosclerotic lesion progression as measured by lesion area (FIG. 9C), acellular area (FIG. 9D), and lipid core area (FIG. 9E). Thus, these results demonstrate the feasibility of therapeutic protocols for atherosclerosis.

Discussion

The results reported herein demonstrate that ABCA1-dependent cholesterol efflux is severely disrupted by the accumulation of free cholesterol in macrophage cells. These results further demonstrate that this disruption parallels a free cholesterol-dependent degradation of the ABCA1 protein. Thus, these results suggest a novel strategy for therapeutic intervention in atherosclerosis, namely the protection of macrophage ABCA1 from free-cholesterol-induced degradation.

Lesional macrophage cells are particularly susceptible to the damaging effects of high levels of intracellular free cholesterol because they internalize large amounts of lipoprotein cholesterol by means other than the LDL receptor, such as by phagocytosis. Therefore, a number of cellular mechanisms for preventing the accumulation of free cholesterol are not available to the macrophage.

Here, it is shown that a free cholesterol-induced degradation of the ABCA1 protein is an early event in the loss of ABCA1-dependent cholesterol efflux activity in free cholesterol-loaded mactophages. It is further demonstrated that this degradation of ABCA1 is proteosome-dependent and occurs well before the appearance of overt biochemical and morphological signs of cytotoxicity, such as a drop in mitochondrial membrane potential, caspase activation, cell shape changes, and membrane permeability disruptions.

At later times, as free cholesterol continues to accumulate, other factors are likely to contribute to the disruption of ABCA1-dependent efflux. For example, alterations in the fluidity of the plasma membrane may adversely affect the transport activity of ABCA1 or decreased cellular ATP levels may contribute to the inactivation of ABCA1, whose transporter activity is ATP-dependent. However, intervention to preserve ABCA1 functionality is less likely to succeed once the cell has sustained this level of damage.

While current efforts to increase ABCA1 activity are focused primarily on increasing ABCA1 gene expression, the results herein suggest that this method will ultimately fail, since the protein will be degraded. Instead, these results point to an alternative strategy, namely the inhibition of the proteosomal degradation of ABCA1 that is demonstrated herein to be induced by excess intracellular free cholesterol.

The results herein also indicate that the triggering of ABCA1 degradation requires trafficking of cholesterol from late endosomes/lysosomes to a peripheral cellular site, perhaps the endoplasmic reticulum, but not to the plasma membrane itself. This interpretation is supported both by the results herein using the NPC1 heterozygous mutant macrophage cells and the results herein with normal macrophages treated with the amphipathic amines imipramine and U18666A. While others have also demonstrated similar effects of low-dose U18666A on cholesterol trafficking to the ER versus the plasma membrane (Underwood et al, 1996), the results presented herein are the first to link this defect with both ABCA1 activity and cholesterol efflux.

Most importantly, the instant work reveals the unexpected discovery that partial inhibition of NPC1, either genetically or pharmacologically, is an effective block against free cholesterol-induced ABCA1 degradation. The surprising result that low concentrations of imipramine and U18666A markedly enhance ABCA1-mediated cholesterol efflux and ABCA1 protein expression in free cholesterol-loaded cells demonstrates that these and similar compounds have therapeutic use as agents in the treatment of atherosclerosis.

Finally, the usefulness of U18666A and related compounds for the treatment of atherosclerosis is demonstrated by the remarkable success of the instant protocol in LDL receptor knockout mice. These results demonstrate that U18666A significantly reduces lesion progression in these mice.

### REFERENCES

1. Ball, R. Y., Stowers, E. C., Burton, J. H., Cary, N. R., Skepper, J. N., and Mitchinson, M. J. (1995) Atherosclerosis 114, 45-54.

2. Bauriedel, G., Schmucking, I., Hutter, R., Luchesi, C., Welsch, U., Kandolf, R., and Luderitz, B. (1997) Z. Kardiol. 86, 902-910.

3. Berberian, P. A., Myers, W., Tytell, M., Challa, V., and Bond, M. G. (1990) Am. J. Pathol. 136, 7180.

4. Brown, M. S. and Goldstein, J. L. (1983) Annu. Rev. Biochem. 52, 223-261.

5. Fuster, V., Badimon, L., Badimon, J. J., and Chesebro, J. H. (1992) N. Engl. J. Med. 326, 242250.

6. Kolodgie, F. D., Narula, J., Burke, A. P., Haider, N., Farb, A., Hui-Liang, Y., Smialek, J., and Virmani, R. (2000) Am. J. Pathol. 157, 1259-1268.

7. Libby, P. and Clinton, S. K. (1993) Curr. Opin. Lipidol. 4, 355-363.

8. Lundberg, B. (1985) Atherosclerosis 56, 93-110.

9. Mallat, Z., Hugel, B., Ohan, J., Leseche, G., Freyssinet, J. M., and Tedgui, A. (1999) Circulation 99, 348-353.

10. Matsuda, K. (1994) Med. Res. Rev. 14, 271-305.

11. Mitchinson, M. J., Hardwick. S. J., and Bennett, M. R. (1996) Curr. Opin. Lipidol. 7, 324-329.

12. Rapp, J. H., Connor, W. E., Lin, D. S., Inahara, T., and Porter, J. M. (1983) J. Lipid Res. 24, 1329-1335.

13. Ross, R. (1995) Annu. Rev. Physiol. 57, 791-804.

14. Tabas, I. (2000) Biochim. Biophys. Acta 1529, 164-174.

15. Tall, A. R. (1998) Eur. Heart J. A31-A35.

16. Underwood, K. W., Andemariam, B., McWilliams, G. L., and Liscum, L. (1996) J. Lipid Res. 37, 1556-68.

17. Williams, K. J. and Tabas, I. (1995) Arterioscler. Thromb. Vasc. Biol. 15, 551-561.

What is claimed is:

1. A method for determining whether an agent increases ABCA1-dependent cholesterol efflux from a cell comprising the steps of:
(a) contacting a free cholesterol-loaded cell with the agent in the presence of a cholesterol acceptor which binds to cholesterol effluxed from a cell via an ABCA1-dependent pathway;
(b) quantitatively determining the efflux of cholesterol from the cell; and
(c) comparing the efflux so determined with a known standard, thereby determining whether the agent increases cholesterol efflux from the cell.

2. The method of claim 1, wherein the cholesterol acceptor of step (a) is selected from the group consisting of apolipoprotein A-I, apolipoprotein A-II, apolipoprotein A-IV, apolipoprotein E, a recombinant apolipoprotein and a synthetic apolipoprotein.

3. The method of claim 2, wherein the cholesterol acceptor of step (a) is apolipoprotein A-I.

4. The method of claim 1, wherein the known standard of step (c) comprises the cholesterol efflux from a free cholesterol-loaded cell in the absence of the agent and in the presence of a cholesterol acceptor.

5. The method of claim 1, wherein the free cholesterol-loaded cell is produced by
(a) contacting a cell with a cholesterol-containing particle, whereby the particle enters the cell, and
(b) contacting the cell with an acyl-CoA-cholesterol acyltransferase inhibitor so as to inhibit the activity of acyl-CoA-cholesterol acyltransferase in the cell,
wherein steps (a) and (b) are performed concurrently or in any other order.

6. The method of claim 5, wherein the cholesterol-containing particle is an acetyl low density lipoprotein.

7. The method of claim 1, wherein (i) the free cholesterol-loaded cell comprises detectably labeled cholesterol and (ii) quantitatively determining the efflux of cholesterol from the cell comprises quantitatively determining the efflux from the cell of the detectably labeled cholesterol.

8. The method of claim 7, wherein the detectable label is a radioisotope.

9. The method of claim 8, wherein the radioisotope is tritium or carbon-14.

10. The method of claim 1, wherein the cell is selected from the group consisting of a macrophage, a hepatic cell and a smooth muscle cell.

11. The method of claim 10, wherein the cell is a macrophage.

12. The method of claim 1, wherein the cell is a human cell.

13. A method for increasing cholesterol efflux from a cell comprising contacting the cell with an agent which increases ABCA1-dependent cholesterol efflux from a cell.

14. A method for decreasing the amount of cholesterol in a cell comprising contacting the cell with an agent which increases ABCA1-dependent cholesterol efflux from the cell.

15. The method of claim 13 or 14, wherein the agent is an inhibitor of an intracellular cholesterol trafficking pathway.

16. The method of claim 15, wherein the intracellular cholesterol trafficking pathway is mediated by a Niemann-Pick C molecule, lysobisphosphatidic acid, and/or lysosomal sphingomyelinase.

17. The method of claim 13 or 14, wherein the cell is selected from the group consisting of a macrophage, a hepatic cell and a smooth muscle cell.

18. The method of claim 17, wherein the cell is a macrophage.

19. The method of claim 13 or 14, wherein the cell is a human cell.

20. The method of claim 13 or 14, wherein the agent is U18666A or a pharmaceutically acceptable salt thereof.

21. The method of claim 20, wherein the agent, when contacted with the cell, is at a concentration of from about 30 nM to about 120 nM.

22. The method of claim 21, wherein the agent, when contacted with the cell, is at a concentration of about 70 nM.

23. The method of claim 13 or 14, wherein the agent is imipramine or a pharmaceutically acceptable salt thereof.

24. The method of claim 23, wherein the agent, when contacted with the cell, is at a concentration of from about 2 μM to about 20 μM.

25. The method of claim 24, wherein the agent, when contacted with the cell, is at a concentration of about 8 μM.

26. A method for increasing the likelihood that a cholesterol-loaded macrophage will survive comprising contacting the macrophage with an agent which increases ABCA1-dependent cholesterol efflux from a macrophage, thereby increasing the likelihood that the macrophage will survive.

27. A method for decreasing the likelihood that a cholesterol-loaded macrophage will contribute to the progression of atherosclerosis in a subject comprising contacting the macrophage with an agent which increases ABCA1-dependent cholesterol efflux from a macrophage, thereby decreasing the likelihood that the macrophage will contribute to the progression of atherosclerosis in the subject.

28. The method of claim 26 or 27, wherein the agent is an inhibitor of an intracellular cholesterol trafficking pathway mediated by a Niemann-Pick C molecule, lysobisphosphatidic acid, and/or lysosomal sphingomyelinase.

29. The method of claim 26 or 27, wherein the agent is U18666A or a pharmaceutically acceptable salt thereof.

30. The method of claim 29, wherein the agent, when contacted with the cell, is at a concentration of from about 30 nM to about 120 nM.

31. The method of claim 30, wherein the agent, when contacted with the cell, is at a concentration of about 70 nM.

32. The method of claim 26 or 27, wherein the agent is imipramine or a pharmaceutically acceptable salt thereof.

33. The method of claim 32, wherein the agent, when contacted with the cell, is at a concentration of from about 2 μM to about 20 μM.

34. The method of claim 33, wherein the agent, when contacted with the cell, is at a concentration of about 8 μM.

35. The method of claim 27, wherein the subject is a human.

36. The method of claim 27, wherein the agent is admixed with a pharmaceutically acceptable carrier.

37. A method for treating a subject afflicted with atherosclerosis comprising administering to the subject a therapeutically effective amount of an agent which increases ABCA1-dependent cholesterol efflux from a cell, thereby treating the subject.

38. The method of claim 37, wherein the cell is a macrophage cell.

39. The method of claim 37, wherein the agent is an inhibitor of an intracellular cholesterol trafficking pathway mediated by a Niemann-Pick C molecule, lysobisphosphatidic acid, and/or lysosomal sphingomyelinase.

40. The method of claim 37, wherein the agent is U18666A or a pharmaceutically acceptable salt thereof.

41. The method of claim 37, wherein the agent is imipramine or a pharmaceutically acceptable salt thereof.

42. The method of claim 37, wherein the subject is a human.

43. The method of claim 37, wherein the therapeutically effective amount of the agent is less than about 3.75 mg of agent per kg of the subject's body weight.

44. The method of claim 43, wherein the therapeutically effective amount of the agent is about 0.75 mg of agent per kg of the subject's body weight.

45. The method of claim 37, wherein the agent is admixed with a pharmaceutically acceptable carrier.

46. An article of manufacture comprising packaging material and a pharmaceutical agent, wherein the pharmaceutical agent increases ABCA1-dependent cholesterol efflux from a cell and wherein the packaging material comprises a label indicating that the pharmaceutical agent is intended for use in treating a subject afflicted with atherosclerosis.

47. The article of claim 46, wherein the cell is a macrophage.

48. The article of claim 46, wherein the agent is an inhibitor of an intracellular cholesterol trafficking pathway mediated by a Niemann-Pick C molecule, lysobisphosphatidic acid, and/or lysosomal sphingomyelinase.

49. The article of claim 46, wherein the agent is U18666A or a pharmaceutically acceptable salt thereof.

50. The article of claim 46, wherein the agent is imipramine or a pharmaceutically acceptable salt thereof.

51. The article of claim 46, wherein the subject is a human.
